(19) United States
(12) Patent Application Publication
(10) Pub. No.: US 2014/0066819 A1
(43) Pub. Date: Mar. 6, 2014

(54) LUMINESCENCE-GUIDED FOCUSED ULTRASOUND APPARATUS AND METHOD

(71) Applicant: Bruker Biospin Corporation, Billerica, MA (US)

(72) Inventor: Gilbert D. Feke, Durham, CT (US)

(73) Assignee: Bruker Biospin Corporation, Billerica, MA (US)

(21) Appl. No.: 13/863,041

(22) Filed: Apr. 15, 2013

Related U.S. Application Data
(60) Provisional application No. 61/623,644, filed on Apr. 13, 2012.

(51) Int. Cl. A61N 7/02

(52) U.S. Cl. CPC A61N 7/02
USPC 601/3

(57) ABSTRACT

A luminescence-guided focused ultrasound apparatus, comprising at least one focused ultrasound transducer that delivers focused ultrasound energy to at least one focal region within the subject, at least one luminescence detector that detects a luminescence signal generated within the subject, and at least one guiding element that guides the at least one focused ultrasound transducer to deliver focused ultrasound energy to the at least one focal region wherein the at least one guiding element is influenced by the detected luminescence signal. The at least one focused ultrasound transducer may be configured in operate in a plurality of modes, including a low-intensity mode for delivering low-intensity focused ultrasound energy and a high-intensity mode for delivering high-intensity focused ultrasound energy.

### CROSS REFERENCE TO RELATED APPLICATIONS

This patent application claims priority from U.S. Provisional Application designated Ser. No. 61/623,644 filed Apr. 13, 2012 which is hereby incorporated by reference.

### FIELD OF THE INVENTION

The present invention relates generally to an apparatus and method of focused ultrasound application and, more particularly, to a luminescence-guided focused ultrasound apparatus and method for delivering focused ultrasound energy to a subject.

### SUMMARY OF THE INVENTION

The present invention relates to a luminescence-guided focused ultrasound apparatus and method for delivering focused ultrasound energy to a subject.

The apparatus comprises at least one focused ultrasound transducer for delivering focused ultrasound energy to at least one focal region within the subject, at least one luminescence detector for detecting a luminescence signal generated within the subject and at least one guiding element for guiding the at least one focused ultrasound transducer to deliver focused ultrasound energy to the at least one focal region wherein the at least one guiding element is influenced by the detected luminescence signal. The at least one focused ultrasound transducer may be configured in one or more modes, including a low-intensity mode for delivering low-intensity focused ultrasound energy and a high-intensity mode for delivering high-intensity focused ultrasound energy. The at least one focused ultrasound transducer may alternatively comprise at least two focused ultrasound transducers, wherein a first of the at least two focused ultrasound transducers is selected to provide a low-intensity mode for delivering low-intensity focused ultrasound energy and a second of the at least two focused ultrasound transducers is selected to provide a high-intensity mode for delivering high-intensity focused ultrasound energy. The at least one luminescence detector may comprise at least two luminescence detectors, wherein a first of the at least two luminescence detectors is configured to detect a luminescence signal, such as that generated by a genetic reporter within the subject, and a second of the at least two luminescence detectors is configured to detect a modulated luminescence signal, such as that generated by focused ultrasound energy, for example low-intensity focused ultrasound energy, that modulates the intensity and/or wavelength spectrum of the luminescence signal, for example by local hyperthermia. The apparatus may further comprise a coupling element positioned between the ultrasound transducer and the subject for transmitting ultrasound energy from the transducer to the subject. The apparatus may further comprise an acoustic window positioned between the coupling element and the subject that allows all or a portion of the focused ultrasound energy to be transmitted from the focused ultrasound transducer to the subject. The acoustic window may comprise a flexible membrane. The flexible membrane may have the form of a sheet. The flexible membrane may alternatively have the form of a bowl. The flexible membrane may alternatively have the form of a flexible membrane tip of a cone which comprises the coupling element, such as a water cone. The flexible membrane may have the form of a thin, clear plastic sheet wrapped around the subject. The subject may be a small animal, such as a mouse or rat, that has a craniocaudal axis. The flexible membrane may alternatively be an elongated flexible membrane for supporting the small animal in a position with its craniocaudal axis transverse to an axis of elongation of the elongated flexible membrane. The apparatus may further comprise a forming mechanism for forming the elongated flexible member into an upwardly open, U-shaped loop, the loop being sized for receiving and engaging the small animal. The apparatus may further comprise a moving mechanism for moving the elongated flexible member in a direction of the axis of elongation while maintaining the U-shaped loop, whereby movement of the elongated flexible member applies torsion to the small animal so that it is rotated about its craniocaudal axis. The at least one luminescence detector may be positioned outside the coupling element. The subject may alternatively be completely or partially submerged within the coupling element. The at least one luminescence detector may be positioned within or submerged within the coupling element. The apparatus may further comprise a processing system for processing input, such as a luminescence signal or modulated luminescence signal, and generating output, such as a three-dimensional map of the luminescence signal.

The method comprises steps of providing an apparatus comprising at least one focused ultrasound transducer, at least one luminescence detector and at least one guiding element, detecting a luminescence signal from a subject, guiding at least one focused ultrasound transducer to deliver focused ultrasound energy to at least one focal region within the subject based on the luminescence signal and delivering focused ultrasound energy using the at least one focused ultrasound transducer to at least one focal region within the subject. The method may further comprise steps of configuring or selecting the at least one focused ultrasound transducer in a low-intensity mode for delivering low-intensity focused ultrasound energy, delivering low-intensity focused ultrasound energy, and scanning the at least one focal region of the at least one focused ultrasound transducer to induce a modulated luminescence signal at a plurality of scan positions, inducing a modulated luminescence signal at a plurality of scan positions to obtain a three-dimensional map of the luminescence signal, and obtaining a three-dimensional map of the luminescence signal by detecting the modulated luminescence signal and correlating the strength of the modulated luminescence signal to the plurality of scan positions. The method may further comprise steps of configuring or selecting the at least one focused ultrasound transducer in a high-intensity mode for delivering high-intensity focused ultrasound energy, guiding the at least one focused ultrasound transducer using the three-dimensional map of the luminescence signal and delivering high-intensity focused ultrasound energy to target at least one focal region within the subject wherein the at least one focal region is guided by the three-dimensional map of the luminescence signal. The method may further comprise steps of configuring or selecting the at least one focused ultrasound transducer to deliver focused ultrasound energy in a low-intensity mode, delivering focused ultrasound energy in a low-intensity mode to at least one focal region to attempt to induce a modulated luminescence signal, detecting the presence or absence of the modulated luminescence signal, configuring or selecting the at least one focused ultrasound transducer in a high-intensity mode and delivering focused ultrasound energy in a high-intensity mode to the at least one focal region based on the presence or absence of the modulated luminescence signal. The method may further comprise steps of delivering focused ultrasound in a high-intensity mode to the at least one focal region based on the strength of the modulated luminescence signal. The method may further comprise providing a coupling element. The method may further comprise providing an acoustic window. The method may further comprise providing an acoustic window in the form of an elongated flexible membrane having an axis of elongation and longitudinally extending edges, forming the elongated flexible membrane into an upwardly open, U-shaped loop, the loop being sized for receiving and engaging the subject, supporting the subject in the U-shaped loop with the craniocaudal axis transverse to the axis of elongation, moving the elongated flexible membrane sequentially in a direction of the axis of elongation while maintaining the U-shaped loop, whereby torsion is applied to the subject so that it is rotated to various angles about its craniocaudal axis, detecting the luminescence signal from the subject at various angles of craniocaudal rotation so as to guide the at least one focused ultrasound transducer to deliver focused ultrasound energy to at least one focal region within the subject based on the luminescence signal

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures.

FIG. 1 is a schematic diagram of an exemplary luminescence-guided focused ultrasound apparatus according with an embodiment of the present invention; and

FIG. 2 is a perspective view of a focused ultrasound system according with an embodiment of the present invention.

### DETAILED DESCRIPTION OF THE INVENTION

A luminescence-guided focused ultrasound apparatus and method are provided. In one beneficial application, the luminescence-guided focused ultrasound apparatus and method can be implemented for delivery of focused ultrasound energy to small animals. It is also recognized, however, that the focused ultrasound apparatus and method described here below is also suitable for delivery of focused ultrasound energy to localized areas of the human body, including areas such as to arms, legs, wrists, and hands and torso. Alternatively this focused ultrasound apparatus and method may also be used on inanimate objects.

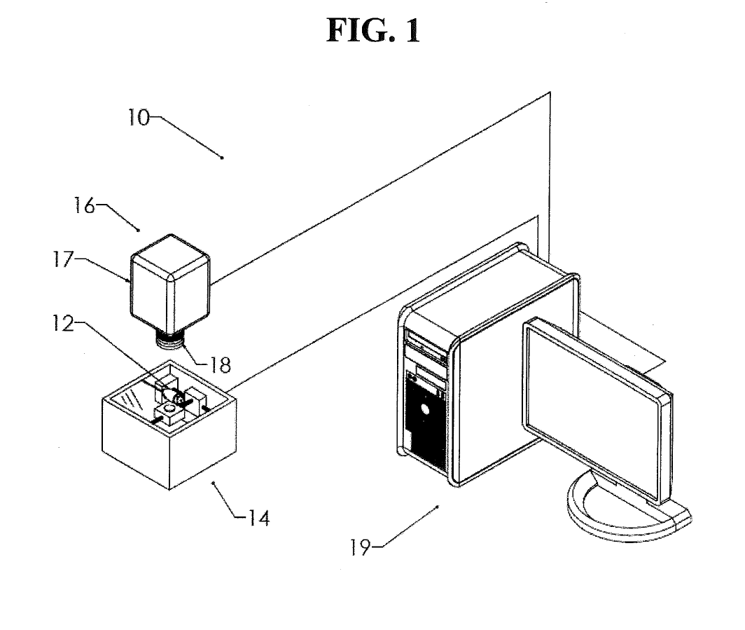

Referring to FIG. 1, an exemplary embodiment of luminescence-guided focused ultrasound apparatus 10 for delivering focused ultrasound energy to a subject 12 or object of interest, which in one application can be a small animal, comprises a focused ultrasound system 14, at least one luminescence detector 16 for detecting a luminescence signal from subject 12, and a guiding element 19. In the embodiment shown, the at least one luminescence detector 16 comprises a camera 17 with a lens 18 mounted thereupon. The camera may comprise a charge coupled device camera, an electron multiplying charge coupled device camera, an intensified charge coupled device camera, or a CMOS camera. The camera may comprise a lightfield camera known to those of ordinary skill in the art as comprising an array of microlenses in combination with a sensor. Alternatively, the at least one luminescence detector may comprise two or more cameras with lenses respectively mounted thereupon, or other devices known to those of ordinary skill in the art, such as photodiodes, avalanche photodiodes, photomultiplier tubes, photon counting devices, and arrays thereof. In the embodiment shown, the guiding element comprises a computer system including a computer that interfaces with both the ultrasound system 14 and the at least one luminescence detector 16.

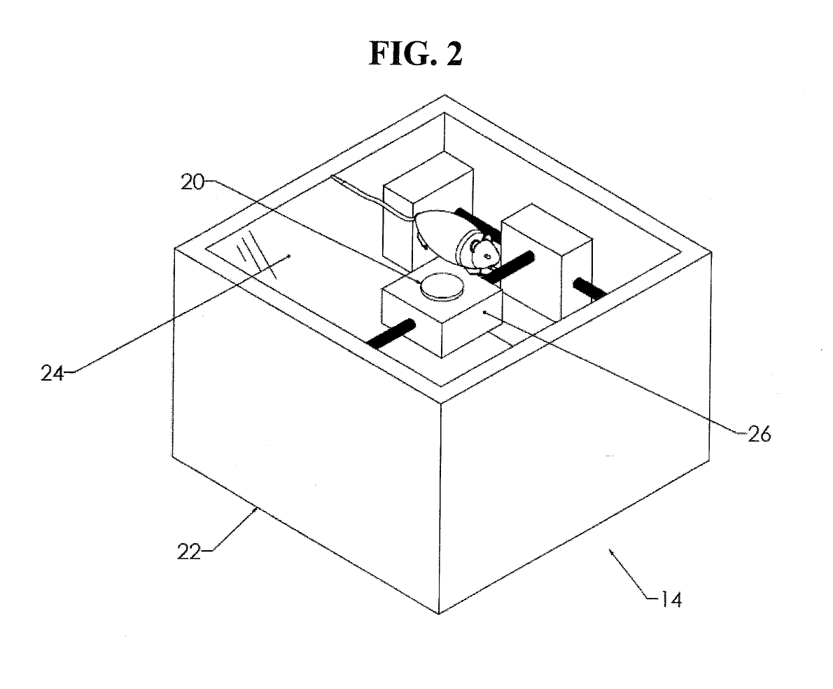

Referring to FIG. 2, focused ultrasound system 14 includes at least one focused ultrasound transducer 20 configured to generate and emit focused ultrasound energy toward subject 12. The focusing can be achieved by mechanical means such as lenses and/or reflectors (not shown), or by manufacturing the at least one focused ultrasound transducer 20 with such a shape that desired focusing will be achieved. Alternatively, the at least one focused ultrasound transducer 20 can be a phased array device capable of electronic focusing and steering of the focal point or region of the ultrasound energy.

Ultrasound energy is emitted by the at least one focused ultrasound transducer 20 when electrical energy is transmitted thereto, as is known in the art to control emission of ultrasound energy. The focused ultrasound energy emitted by the at least one focused ultrasound transducer 20 is transmitted via a coupling element 22 positioned between the at least one focused ultrasound transducer 20 and the subject 12. In one embodiment, coupling element 22 comprises a water bath (i.e., degassed water) in which the at least one focused ultrasound transducer 20 is submerged such that the generated focused ultrasound energy will be directed therethrough. An acoustic window 24 is positioned between the water bath 22 and the subject 12 that allows all or a portion of the focused ultrasound energy to be transmitted from the at least one focused ultrasound transducer 20 to the target subject as shown in FIG. 2.

While the water bath 22 is shown and described in FIG. 2 as comprising the coupling element, it is also envisioned that other low attenuation media/mechanisms can comprise the coupling element 22, such as ultrasound gel or another solid, or a flexible water or other liquid coupling. Additionally, while shown in FIG. 2 as being positioned below the subject 12, it is envisioned that the at least one focused ultrasound transducer 20 can be located relative to the subject in any of a plurality of positions. Thus, in one embodiment, the at least one focused ultrasound transducer 20 can be located above subject 12, such as in a water cone with a flexible coupling (e.g., flexible membrane tip) to the target region of the subject. In another embodiment, the at least one focused ultrasound transducer 20 can be mounted on the side of subject 12 with a membrane coupling the transducer to the subject. In any of these arrangements, the acoustic coupling can be achieved by direct contact of the at least one focused ultrasound transducer 20 with the subject 12 or with a coupling element 22 such as ultrasound gel or a flexible water coupling. In another embodiment, acoustic window 24 may comprise a thin plastic film wrapped substantially around the subject 12, and acoustic window 24, containing subject 12 therein, may be submerged in water bath 22. Further, the at least one focused ultrasound transducer 20 may comprise a plurality of focused ultrasound transducers, wherein the plurality may be configured to deliver focused ultrasound energy to the same or different focal regions within subject 12.

Referring still to FIG. 2, included in focused ultrasound system 14 is a positioning system 26 configured to control the position of the at least one ultrasound transducer 20 for precise positioning thereof relative to subject 12. Positioning system may comprise elements known to those of ordinary skill in the art, such as stepper motors, DC motors, ultrasonic motors, encoders, lead screws, linear stages, and the like. Positioning system 26 may be positionable in one, two, or three dimensions. Positioning system 26 may be positioned by translation, rotation, or a combination thereof. For example, positioning system 26 may be configured to allow the at least one focused ultrasound transducer 20 to be moved in a horizontal plane defined by the x axis and y axis, as well as vertically out of this plane, along the z axis. It is further envisioned, however, that positioning system 26 can provide a greater or lesser number of degrees of freedom. Positioning system 26 may allow four or five degrees of freedom by enabling rotation of focused ultrasound transducer 20 about one or both of the x and y axes (e.g., by addition of a rotational-type ultrasonic motor), or conversely, may be limited to only two degrees of freedom by limiting linear translation to the horizontal plane. The amount of translation provided by positioning system 26 along each of the x, y, and z axes can also vary. When focused ultrasound system 14 is designed for use with focused ultrasound experiments on small animals, for example, travel length of the position system 26 can be limited to 5 cm in the horizontal and 2.5 cm vertical. It is envisioned, however, that the travel length provided by positioning system 26 may be extended to over 20 cm when focused ultrasound system 14 is designed for delivery of focused ultrasound to localized areas of the human body. As will be further explained below, positioning system 26 functions under the control of guiding element 19 to position the at least one ultrasound transducer 20 in a desired location relative to at least one target location 28 that is identified in subject 12 by way of information derived by guiding element 19 from the detected luminescence signal. That is, guiding element 19 processes the detected luminescence signal data, from which at least one target location is identified. While guiding element 19 is shown in FIG. 1 as being a unitary element shared by luminescence detector 16 and focused ultrasound system 14, it is recognized that guiding element 19 may be distributed among more than one subsystem, for example a control system for the luminescence detector 16 in communication with a control system for focused ultrasound system 14.

The at least one luminescence detector 16 is configured to be useful for detecting a luminescence signal from subject 12. The luminescence signal comprises at least information about the location of the signal within subject 12. The luminescence signal may further comprise information about the intensity of the signal. Unlike known systems and methods which use detectors that provide signals based on anatomical information (e.g., MRI, X-ray, computed tomography) or exogenous radiolabled contrast agents (e.g., PET, SPECT) to guide the delivery of focused ultrasound energy, the present invention provides the advantage that the signal may be based on bioluminescence from a genetic reporter, for example a luciferase enzyme that uses luciferin as a substrate that is administered to the subject 12 to generate direct light emission (i.e., luminescence) or downshifted wavelength light emission via bioluminescence resonant energy transfer in which an acceptor construct such as a molecule or nanoparticle emits light donated by the luciferin reaction. The present invention is advantageous in that genetic reporters in general provide signals with high biomolecular sensitivity and specificity. Unlike the signals provided by exogenous contrast agents, the signals generated by genetic reporters are localized within the realms and control of a biomolecular tertiary structure as known to those of ordinary skill in the art. Among genetic reporters, luciferase based genetic reporters provided an additional advantage because, unlike fluorescent genetic reporters, there is no autofluorescence caused by excitation light interacting with tissue outside the realms and control of the biomolecular tertiary structure. In one application, the luminescence detector 16 may detect a luminescence signal from a small tumor expressing luciferase, where the tumor may otherwise be undetectable by any other method of detection. The guiding element 19 may then control positioning system 26 to guide the delivery of focused ultrasound energy to the location of the detected luminescence signal to induce local hyperthermia at the tumor that emits the luminescence signal (e.g., for a therapeutic effect). In another application, the luminescence detector 16 may detect a luminescence signal from tissue expressing luciferase so as to provide information to guiding element 19 to control positioning system 26 to induce local hyperthermia in the tissue for the purpose of promoting expression of proteins.

In operation, guiding element 19 receives luminescence signal data from the at least one luminescence detector 16 and, based on the luminescence signal data, identifies at least one target location. The guiding element 19 is programmed to determine at least one positional coordinate of the at least one target location within a coordinate system of the luminescence detection space. The positional coordinate is then registered with a coordinate system of the positioning system 26. Once registered with a coordinate system of the positioning system 26, the at least one positional coordinate of the at least one target location is sent as an input signal to the positioning system 26. The positioning system 26 receives the at least one positional coordinate and is actuated by the guiding element 19 to position the at least one ultrasound transducer 20 to deliver focused ultrasound energy to the at least one target location. That is, the positioning system 26 moves the at least one ultrasound transducer 20 such that the focal point of the ultrasound energy generated by the transducer, once energized, will match the at least one positional coordinate of the at least one target location. The at least one target location is thus sonicated by the at least one ultrasound transducer 20.

Beneficially, the positioning system 26 is controlled by the guiding element 19 to allow for the continuous repositioning of the at least one ultrasound transducer 20 during luminescence signal detection. This positioning control of the at least one ultrasound transducer 20 (via positioning system 26) during luminescence signal detection allows for the delivery of focused ultrasound energy to a plurality of target locations or points in rapid succession, such that a plurality of target points in a region within the subject 12 can be sonicated/exposed within a short timeframe. Thus, via the control of the guiding element 19, the at least one ultrasound transducer 20 is positioned to deliver focused ultrasound energy to a plurality of target points within the subject 12 in any of a point exposure, scanned linear (1D, 2D, 3D) exposure, or raster scan (1D, 2D, 3D) exposure pattern, or along another continuous trajectory.

According to an embodiment, the guiding element 19 is programmed to control the positioning system 26 and the at least one ultrasound transducer 20 so as to cause the ultrasound transducer to sequentially emit beams of focused ultrasound energy according to an interleaved sonication pattern. That is, the guiding element 19 is programmed to control the positioning system 26 to position the at least one ultrasound transducer 20 to a series of locations, and for the at least one ultrasound transducer 20 to sequentially emit beams of focused ultrasound energy at each of the series of locations such that each of the locations is sonicated within a specified repetition period. In other words, if a target point requires a sonication every two seconds, the system can scan quickly to multiple points such that each point is exposed every two seconds. The guiding element 19 is thus programmed to define a repetition period for emitting beams of focused ultrasound energy from the at least one ultrasound transducer 20 to a plurality of target locations. The guiding element 19 is further programmed to cause the motor controller to control the drive apparatus to sequentially position the ultrasound transducer, such that the beams of focused ultrasound energy are localized within each of the target locations within the repetition period.

In another embodiment, a luminescence-guided focused ultrasound apparatus is comprised of a first at least one luminescence detector for detecting a luminescence signal in at least one general location within a subject, a guiding element for guiding a positioning system to position an at least one focused ultrasound transducer for scanning a focal region of low-intensity focused ultrasound energy generated by the at least one focused ultrasound transducer within the general location for modulating the luminescence signal, and a second at least one luminescence detector for detecting a modulated luminescence signal resulting from the interaction of the scanned focal region with the luminescence signal for obtaining a three-dimensional map of the luminescence signal The three-dimensional map of the luminescence signal is obtained by a signal processing system which correlates the absence, presence or strength of the modulated luminescence signal with the position of the focal region of the delivered focused ultrasound energy. The embodiment may further comprise the guiding element for guiding the positioning system to position the at least one focused ultrasound transducer for targeting a focal region of high-intensity focused ultrasound energy generated by the at least one focused ultrasound transducer based on the three-dimensional map of the luminescence signal, for example to induce local hyperthermia at a tumor in a subject that emits the luminescence signal (e.g., for a therapeutic effect).

The invention has been described with reference to preferred embodiments, however, it will be appreciated that variations and modifications can be effected by a person of ordinary skill in the art without departing from the scope of the invention.

What is claimed is:

1. A luminescence-guided focused ultrasound apparatus, comprising:
at least one focused ultrasound transducer that delivers focused ultrasound energy to at least one focal region within the subject;
at least one luminescence detector that detects a luminescence signal generated within the subject;
at least one guiding element that guides the at least one focused ultrasound transducer to deliver focused ultrasound energy to the at least one focal region wherein the at least one guiding element is influenced by the detected luminescence signal,
where the at least one focused ultrasound transducer is configured in operate in a plurality of modes, including a low-intensity mode for delivering low-intensity focused ultrasound energy and a high-intensity mode for delivering high-intensity focused ultrasound energy.

2. The apparatus of claim 1, where the at least one focused ultrasound transducer may alternatively comprise at least two focused ultrasound transducers, where a first of the at least two focused ultrasound transducers is selected to provide a low-intensity mode for delivering low-intensity focused ultrasound energy and a second of the at least two focused ultrasound transducers is selected to provide a high-intensity mode for delivering high-intensity focused ultrasound energy.

3. The apparatus of claim 1, where the at least one luminescence detector may comprise at least two luminescence detectors, wherein a first of the at least two luminescence detectors is configured to detect a luminescence signal, such as that generated by a genetic reporter within the subject, and a second of the at least two luminescence detectors is configured to detect a modulated luminescence signal, such as that generated by focused ultrasound energy.

4. The apparatus of claim 1, further comprising a coupling element positioned between the ultrasound transducer and the subject for transmitting ultrasound energy from the transducer to the subject.

5. The apparatus of claim 4, further comprising an acoustic window positioned between the coupling element and the subject that allows all or a portion of the focused ultrasound energy to be transmitted from the focused ultrasound transducer to the subject.

6. The apparatus of claim 5, wherein the acoustic window comprises a flexible membrane.

7. The apparatus of claim 4, wherein the flexible membrane comprises the form of a bowl.

8. The apparatus of claim 4, wherein the flexible membrane comprises an elongated flexible membrane for supporting the small animal in a position with its craniocaudal axis transverse to an axis of elongation of the elongated flexible membrane.

9. The apparatus of claim 8, the apparatus may further comprise a forming mechanism for forming the elongated flexible member into an upwardly open, U-shaped loop, the loop being sized for receiving and engaging the small animal.
